(19) United States
(12) Patent Application Publication
(10) Pub. No.: US 2005/0100580 A1
(43) Pub. Date: May 12, 2005

(54) Hydrophilic coated medical device

(75) Inventors: Thomas A. Osborne, Bloomington, IN (US); Andrew Hoffa, Bloomington, IN (US); Bruce Gingles, Bloomington, IN (US); Jason Urbanski, Ellettsville, IN (US)

Correspondence Address: BRINKS HOFER GILSON & LIONE/CHICAGO/COOK, PO BOX 10395, CHICAGO, IL, 60610, US

(73) Assignee: Cook Incorporated

(21) Appl. No.: 10/965,584

(22) Filed: Oct. 14, 2004

Related U.S. Application Data
(60) Provisional application No. 60/511,397, filed on Oct. 14, 2003.

(51) Int. Cl. A61F 2/02; A61F 2/06

(52) U.S. Cl. 424/424; 427/214; 623/1.42; 623/1.46

(57) ABSTRACT

A medical device comprises a main body, a therapeutic agent and a radiation-curable hydrophilic coating. The hydrophilic coating allows for easy insertion of medical devices, which may include catheters, cannulae, stents, wire guides, and the like. The medical device may include more than one therapeutic agent.

This application claims the benefit of the filing date under 35 U.S.C. § 119(e) of Provisional U.S. Patent Application Ser. No. 60/511,397, filed on Oct. 14, 2003, which is hereby incorporated by reference in its entirety.

### BACKGROUND OF THE INVENTION

The present invention relates generally to medical devices, and more particularly to medical devices that are at least partially implantable into a human or veterinary patient. In preferred embodiments, the invention relates to catheters, cannulae, and medical devices with therapeutic agents and with coatings.

It has become common to treat a variety of medical conditions by introducing an implantable medical device partly or completely into a portion of the body, such as a vessel. For example, many treatments of the vascular system entail the introduction of medical devices, such as stents, catheters, balloons, wire guides, cannulae, and the like, into a vessel, such as an artery or vein. The device utilized may serve a variety of purposes, such as maintaining vessel patency, providing access to a body portion, and delivering one or more therapeutics. Devices such as cannulae may also be used in the biliary, urinary, renal, or gastroenteric systems.

During introduction and/or implantation of these devices, however, adverse affects can occur. For example, the vessel walls can be disturbed or injured during navigation of the device through the vessel. As a result, clot formation or thrombosis can occur at the site of injury, which may cause stenosis or occlusion of the vessel. Moreover, if the device is left within the patient for an extended period of time, a thrombus often forms on the device itself, which may also lead to stenosis or occlusion of the vessel. These conditions may place the patient at risk of a variety of complications, including heart attack, pulmonary embolism, and stroke. Thus, the use of such a medical device can include the risk of causing precisely the problems that its use was intended to ameliorate.

Implantable medical devices also present an opportunity for the establishment of infection. Microorganisms may colonize the device and establish an infection at the implant site, which may cause injury or illness and may even destroy the functionality of the device. The risk of infection is particularly acute for partially implanted medical devices, percutaneously introduced into the vascular system of a patient for long term use, such as hemodialysis and drug infusion catheters. These devices are exposed to both the external and internal environments, providing a link between these two very different environments. Microorganisms can use the device to gain access to the internal environment, ultimately colonizing and possibly establishing an infection. Indeed, the occurrence of infection with indwelling catheters is a common problem that can necessitate repeated removal and replacement of catheters, in addition to treatment of infections.

The art contains many examples of devices adapted to inhibit or prevent such infections. For example, U.S. Pat. No. 4,677,143 to Lavrin describes an antimicrobial coating placed on the exterior of a medical device, such as a catheter. Also, U.S. Pat. No. 3,598,127 to Wepsic describes a device with an antimicrobial placed as a powder in the device and surrounded by a permeable layer. Furthermore, devices are known that include more than one therapeutic agent. For example, U.S. Pat. No. 5,820,607 to Tcholakia describes a layered catheter that includes an intermediate layer surrounded by a permeable layer. The intermediate layer can include multiple therapeutic agents. Also, U.S. Pat. No. 4,999,210 to Solomon describes a layered device that can include different therapeutic agents in different layers.

Despite the many advantages of catheters and other in-dwelling devices, their use is not without disadvantages besides possible infections. For instance, it would be desirable to reduce the size of the device or catheter to be used. Typically, in order to implant certain of these devices, the walls of the devices are made a little thicker, and the outer diameter of the device is a little greater, in order to impart a desired stiffness or flexural modulus, to the device. This greater stiffness allows the surgeon or operating team member to place the catheter or cannula in the desired location in the patient by allowing a little more force to be applied for the placement. It would be desirable to minimize the size of the device and the force necessary to place the device. It would also be desirable to minimize the trauma caused upon insertion into to the desired site.

### SUMMARY OF THE INVENTION

The present invention provides a medical device that includes a therapeutic agent and a hydrophilic coating. One embodiment of the invention is a medical device for at least partial implantation in a patient, comprising an elongated member, a therapeutic agent disposed on an exterior of the elongated member, and a hydrophilic coating covering the therapeutic agent and the exterior.

Another aspect of the invention is a method of making a medical device. The method comprises forming a tubular member defining at least one lumen, coating at least an exterior surface of the tubular member with a radiation-curable coating, and curing the coating.

Another aspect of the invention is a medical device for at least partial implantation in a patient. The device comprises a tube member defining a lumen, a mixture of rifampin and minocycline distributed throughout at least a portion of the tube, and a radiation-curable hydrophilic coating on an exterior of the device.

Another aspect of the invention is a method of making a medical device. The method comprises forming a tubular member defining at least one lumen, coating at least an exterior surface of the tubular member with a radiation-curable coating, and curing the coating. The coating comprises a reagent useful as a surface coating agent, the reagent having a nonpolymeric core molecule comprising an aromatic group, the core molecule having attached thereto, either directly or indirectly, one or more substituents comprising negatively charged groups, and two or more photoreactive species attached to the core molecule through one or more spacer groups, wherein the negatively charged groups are independently selected from salts of organic acids, the organic acids are selected from sulfonic acid, carboxylic acid, and phosphoric acid, the aromatic group is a benzene radical, the photoreactive species are aryl ketones that may be the same or may be different, and the spacer groups each independently comprise a radical of the formula —O—(CH₂)ₙ—, wherein n is a whole number equal to at least one.

There are many ways to practice the present invention, a few of which are shown in the following drawings and specification. The embodiments described below are not meant to limit the invention, but rather to describe and illustrate the many ways that the present invention may be used.

### BRIEF DESCRIPTION OF THE DRAWINGS

FIG. 1 is a schematic illustration of a medical device according to an embodiment of the present invention.

FIGS. 1A, 1B, 1C and 1D illustrate various cross-sectional shapes and lumen configurations for devices according to the present invention.

FIG. 2 is a schematic illustration of a medical device according to the present invention transcutaneously implanted into a body.

FIG. 3 is a schematic illustration of a medical device according to the present invention implanted subcutaneously into a body.

FIG. 4 is a schematic illustration of a medical device according to an embodiment of the invention.

FIG. 5 is a schematic illustration of a medical device according to an embodiment of the invention.

### DETAILED DESCRIPTION OF THE INVENTION

The present invention provides a medical device with a therapeutic agent and with a photo-reactive hydrophilic coating for easing the entry of the device into the body. The therapeutic agent is positioned in the device by any method ordinary used to provide a therapeutic coating, such as an antibiotic, antimicrobial, or antibacterial coating. To be associated with an elongated member, a therapeutic agent can be applied to a surface of the member, such as by spraying, dipping, coating, dispersal in the base material of the member, e.g., bulk distribution, or any desired method. Indeed, any suitable technique for placing a therapeutic agent in, on, or near a medical device for delivery through the device may be utilized.

The invention is suitable for any medical device in which the therapeutic agent may be utilized and in which there may be an advantage from a reduced size or a reduced force required for insertion into the human or veterinary patient. The invention is particularly well suited for devices used for the delivery of one or more therapeutic agents. Examples of types of devices that can be made in accordance with the present invention include stents, catheters, cannulae, balloons, and bladders. The device need only be at least partially implantable in a patient.

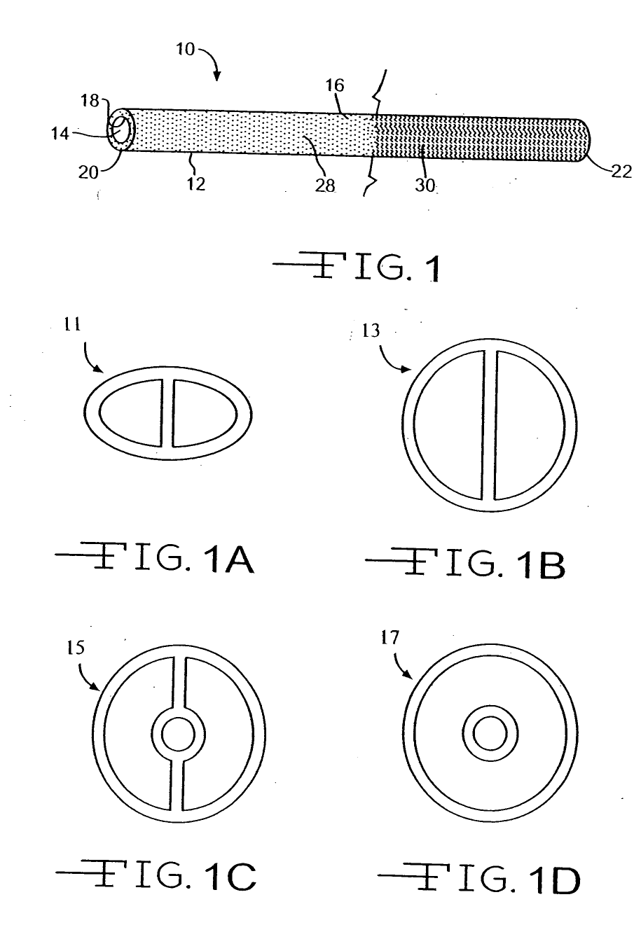

FIG. 1 illustrates a broken cross-sectional view of a medical device according to one embodiment of the present invention. In this embodiment, the medical device 10 comprises a cannula having a main body 12 and defining a lumen 14. The cannula 10 has exterior 16 and interior 18 surfaces, a first or distal end 20, and a second or proximal end 22. The length of the cannula extends from the first end 20 to the second end 22. A therapeutic agent 28 is associated with the cannula 10. The therapeutic agent 28 can be associated with cannula 10 in a variety of manners, mentioned above, by one or more methods of coating or by integration into the base material of the cannula or device. Cannula 10 is also coated in its entirety with a hydrophilic coating 30. In FIG. 1, coating 30 is broken away in distal end 20 in order to portray therapeutic agent 28. Therapeutic agent 28 will be present along the entire length of cannula 10 and coating 30 will cover therapeutic agent 28.

The elongated member can be made from any suitable material. The material need only be acceptable for use in a medical device, i.e., biocompatible and acceptable for the intended use of the device. Preferably, the material is able to have one or more therapeutic agents associated with it. Examples of suitable materials include materials commonly used in medical devices, such as polymers, including silicone and urethane compounds, copolymers, plastics, and metals. The material chosen will depend on several factors, including the intended use of the device, the therapeutic agent or agents that will be used in the device, the ability of the material to have one or more of the agents associated with it, the permeability of the material to the therapeutic agents, and the ability of the material to be formed into members permeable to the therapeutic agents. Other materials useful include polyethylene, polypropylene, polyvinylchloride, and fluoride-containing polymers, such as PTFE.

Silicone is a preferred material for use in one or all of the elongated members of the medical devices according to the present invention. Silicone is preferred for several reasons, including its widespread use in a variety of medical devices, its known biocompatibility, its permeability to numerous sizes, shapes, and types of therapeutic agents, and its ability to associate with therapeutic agents by coating, bulk distribution, and combinations of these approaches. Furthermore, silicone is particularly preferred because it enables the use of bulk distribution methods involving relatively low temperatures, as compared to the higher temperatures needed in methods using thermoplastics and other materials. The use of these relatively low temperatures minimizes damage to the therapeutic agents being distributed within the material. Also, silicone is readily available from a variety of commercial sources in various forms, including powder form which can be readily used in bulk distribution methods. Urethane is also a preferred material.

It has been discovered through testing that hydrophilic coatings ease the entry into the body of medical devices, such as cannulae, catheters, stents, and the like. It has been further discovered that photo-reactive coatings are particularly useful in achieving hydrophilicity. It may be that such coatings more readily fill in minute gaps in surfaces, or it may be that the process of radiation curing, or photoreactive curing, better binds the coating to the surface of the medical device.

When medical devices are coated with medications, the surface of the device tends to be rougher or tackier with the medication or therapeutic agent. The medication may be an antibiotic coating, an antimicrobial coating, antibacterial coatings, antivirals, antiproliferatives, antithrombotics, antimitotics, proteins, nucleic acids, carbohydrates, conjugates, small molecules, and antibodies. While the device will be more effective as a result of the therapeutic agent, the device may also be slightly greater in diameter, and therefore slightly more difficult to insert into the human or veterinary patient. As mentioned above, the greater effort required to insert these devices may require a slightly stiffer, and therefore slightly larger device.

Using UV-curable coatings with excellent hydrophilicity, it has been found that catheters may be reduced in diameter as much as about 0.007 inches, or about 0.5 Fr. Any reduction in diameter is a significant achievement, since it allows for less trauma to the patient and greater patient comfort. Many of these devices are meant for long-term in-dwelling, and any reduction in size while maintaining the same lumen is helpful to the patient.

The coatings may be referred to as UV (ultra-violet light)-curable, radiation-curable, photoreactive, photoimmobilizing, or by other terms. The coatings have in common at least one photoreactive species. Coatings are made from these species and medical devices are then coated. The coatings may be placed via dipping, spraying, or other convenient process, followed by curing the coating. Particularly useful are coatings commercially available from SurModics, Inc., Eden Prairie, Minn., under the trade mark “PhotoLink®.” These coatings are used by the assignee of the present application under the trade name “EZ-Pass.”

The cross-sectional shape of the medical device can be any shape suitable for the types of procedures in which the device will be utilized. A circular cross-sectional shape is particularly preferable in embodiments in which the device comprises a cannula, such as that illustrated in FIG. 1. A circular cross-sectional shape maximizes space within the lumen 14 of the cannula 10 while also providing a suitable shape for interfacing with a body vessel. Furthermore, the medical device can have any suitable configuration of lumen(s), and the chosen configuration will depend on the application for which the device is used. Single and multi-lumen configurations can be utilized. FIGS. 1A, 1B, 1C and 1D illustrate various suitable cross-sectional shapes and lumen configurations for use in medical devices 11, 13, 15, and 17 according to the present invention. While single-lumen and double-lumen applications may be numerous, three-lumen devices are also contemplated, such as a triple-lumen central venous access catheter. Other non-vascular applications may include biliary drainage catheters, gastrostomy catheters, nephrostomy catheters, and suprapubic urinary drainage catheters.

A wide variety of therapeutic agents can be utilized in the present invention. Examples of suitable types of therapeutic agents include antimicrobials, antivirals, antiproliferatives, antithrombotics, antimitotics, proteins, nucleic acids, carbohydrates, conjugates, small molecules, and antibodies. The actual types of agents chosen will depend upon the clinical situation being treated or addressed by the medical device of the invention. The therapeutic agents can be of the same or different types.

Two or more therapeutic agents may be utilized in the medical devices of the invention. Thus, if a suitable difference in diffusion rates exist, two or more derivatives of a therapeutic agent can be utilized. Also, a therapeutic agent and one or more derivatives of the agent can be used. Of course, two completely different therapeutic agents can also be used, so long as a suitable difference in diffusion rates exist between the agents.

Preferably, the therapeutic agents are agents conventionally used in combination therapy. Particularly preferable, the therapeutic agents are agents commonly used in the treatment, inhibition, and/or prevention of microbial infections. Rifampin and minocycline are a particularly preferred pair of therapeutic agents for use in the medical devices according to the present invention.

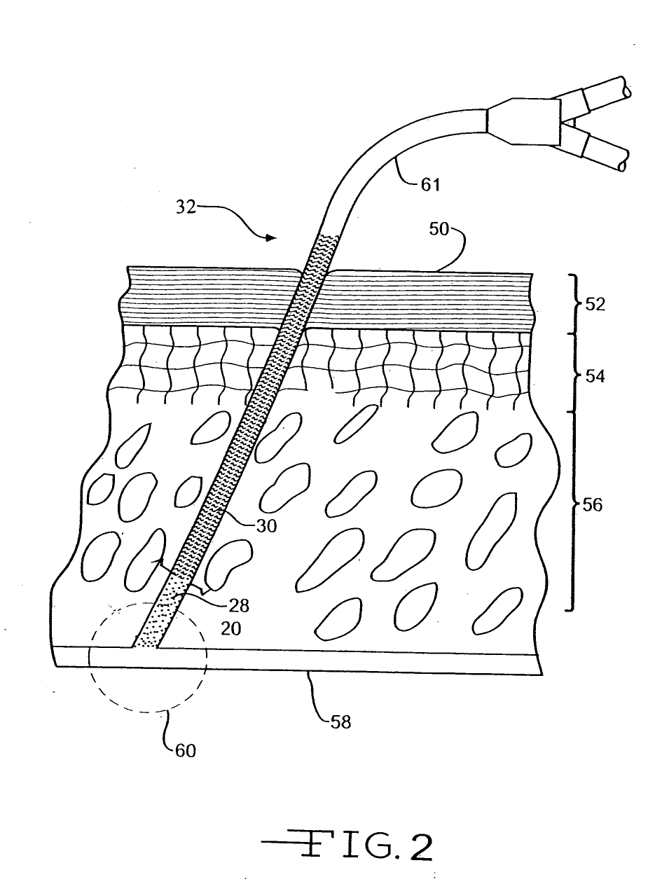

The medical devices according to the present invention can be completely implanted within the body, or only partially implanted within the body. In each scenario, however, at least a portion of the second section of the device remains within the subcutaneous space. FIG. 2 illustrates a schematic of a medical device 32 according to the present invention that is transcutaneously implanted into a body. In this embodiment, the medical device 32 traverses the skin through the epidermis 52, derma 54 and subcutaneous 56 layers to a vessel 58. An interface 60 is formed between the vessel 58 and the device 32. The interface defines a communicative passageway between the vessel 58 and the lumen of device 32. The interface 60 can be a direct insertion of the distal end 20 of device 32 into the vessel 58, or can comprise an attachment of the distal end 20 to vessel 58, such as an anastomosis.

Because device 32 is implanted transcutaneously, device 32 in this embodiment includes a portion 61 that remains external to the body. This portion 61 provides the desired access to the lumen which is in communication with vessel 58. Thus, in this embodiment, vessel 58 can be accessed without further disruption to the skin 50. FIG. 2 depicts coating 30, which extends the length of device 32, as well as therapeutic agent 28.

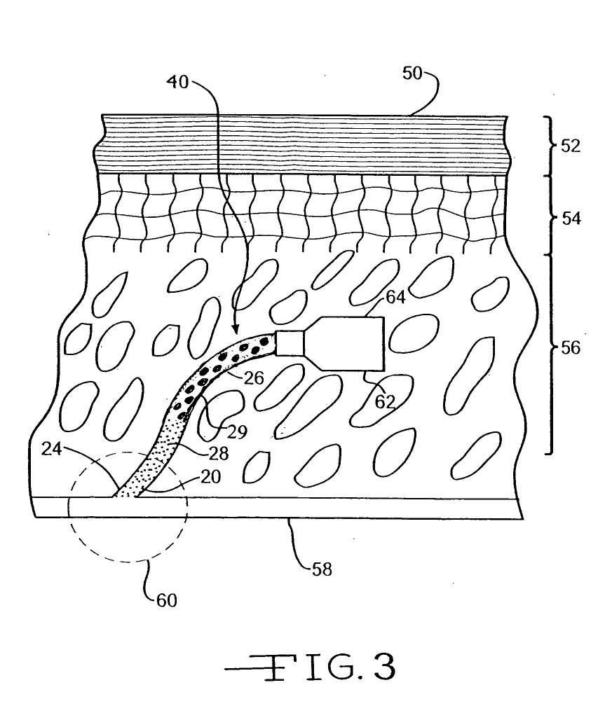

FIG. 3 illustrates a cannula 40 according to the present invention that is completely and subcutaneously implanted within a body. Cannula 40 is divided into two parts, a first part 24 coated with a first therapeutic coating 28 and a second part 26, coated with a second therapeutic coating 29. In this embodiment, cannula 40 also includes an access port 62. The access port 62 defines a chamber that can receive a communicative member, such as a needle, for either withdrawing fluid from or directing fluid into the vessel 58. The skin comprises the epidermis 52, derma 54 and subcutaneous 56 layers, leading to a vessel 58. Typically, the access port 62 includes a section of resealable material 64 that prevents escape of fluid from the cannula 40 when a communicative member is not received by the access port 62. The resealable material can comprise silicon or any other suitable material. Also visible in FIG. 3 is interface 60. Not shown is the coating 30 that is applied after the first and second therapeutic agents 28, 29.

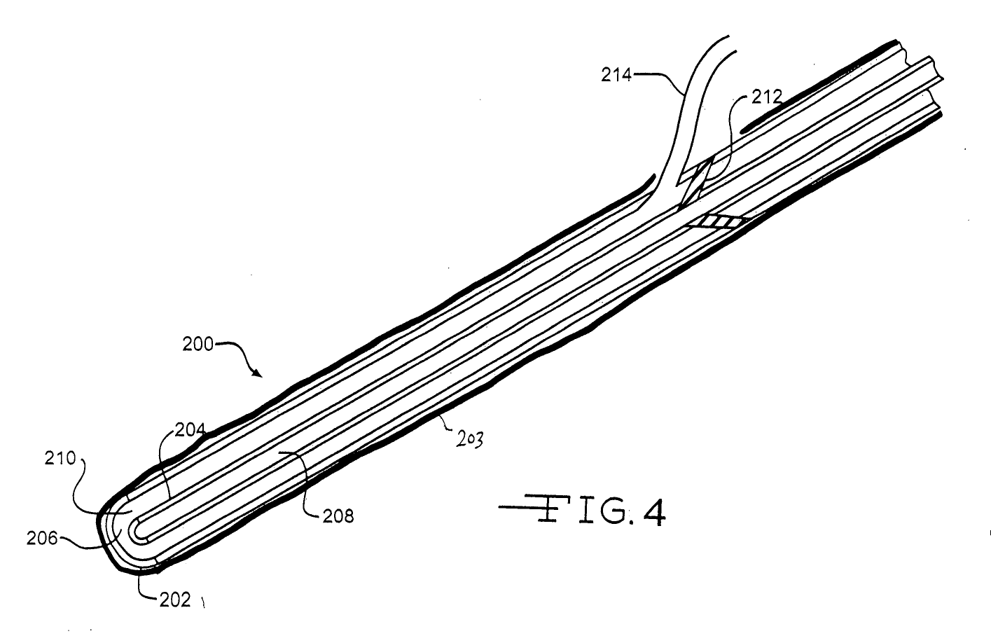

FIG. 4 illustrates a medical device according to another embodiment of the present invention. The medical device according to this embodiment comprises a cannula 200 and includes first 202 and second 204 tubes, the first tube 202 coated with radiation-curable coating 203. The second tube 204 is positioned within a lumen 206 of the first tube 202. The second tube 204 also defines a lumen 208. An annular space 210 is formed between the interior surface of the first tube 202 and the exterior surface of the second tube 204. An access line 214 provides communication with the annular space 210. A seal 212 is positioned proximal to the access line 214 and prevents fluid within the annular space 210 from moving up the cannula away from the body. In this embodiment, the first cannula 202 is preferably porous and a first therapeutic agent is preferably contained within the annular space 210 and escapes from the annular space 210 through the first tube 202 due to its porosity. The access line 214 allows for replacement of the first therapeutic agent that has escaped from the annular space 210 through the first tube 202. A seal (not illustrated) can close the annular space 210 at the distal end of the device 200 to prevent escape of the first therapeutic agent through the distal end. A second therapeutic agent can be placed in the annular space 210 proximal to the seal 212, thereby being separated from the first therapeutic agent. Similar to the first therapeutic agent, the second therapeutic agent will escape from the annular space 210 through the first tube 202 due to its porosity. Alternatively, the second therapeutic agent can be coated onto one or more surfaces of the first 202 and/or second 204 tubes. The lumen 208 of the second tube 204 is placed in communication with a body vessel. This double tube structure allows for the establishment of access to a body vessel and for the replenishment of the first therapeutic agent, which facilitates the use of the medical device as an indwelling cannula.

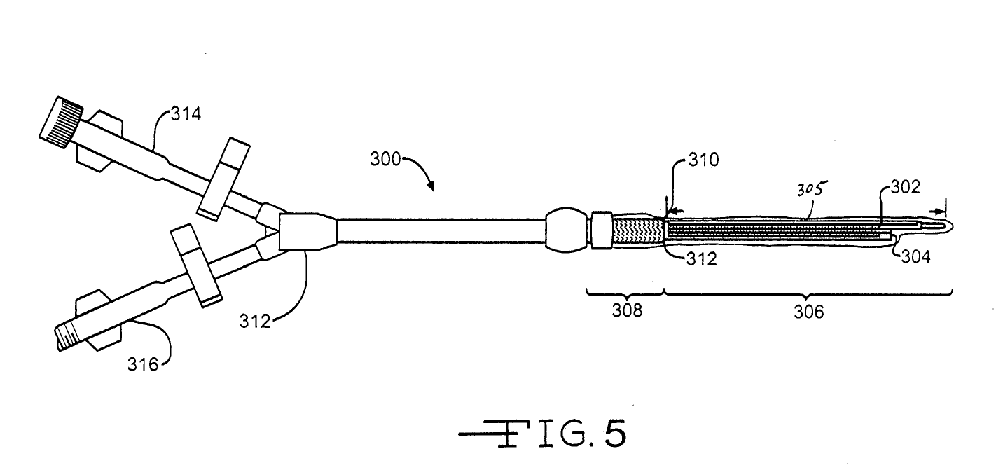

FIG. 5 illustrates a medical device according to another embodiment of the invention. In this embodiment, the medical device comprises a catheter 300 that includes first 302 and second 304 lumens. A first section 306 of the catheter 300 is coated with paclitaxel, and a second section 308 is coated with a blend of rifampin and minocycline. First and second sections 306, 308 are coated with hydrophilic coating 305, preferably an ultra-violet curable coating. In this embodiment, the separator 310 comprises a visual distinction between the first 306 and second 308 sections. Also, the separator 310 defines a slight increase in the diameter of the medical device. The separator 310 includes a taper 312 from the smaller diameter of the first section 306 to the larger diameter of the second section 308. The extracorporeal portion 312 of the catheter includes various connectors 314, 316 that are in individual communication with the first 302 and second 304 lumen, respectively. This device will be easy to insert into the patient and will desirably gradually release the therapeutic agents over a period of several weeks.

Embodiments of the invention include medical devices with a therapeutic agent. In these embodiments, the medical devices are preferably devices suitable for partial implantation in a body. Preferably, the devices have a therapeutic agent in or on a section of the device that will be implanted in the body. For example, a hemodialysis catheter can be coated with an antiproliferative agent, such as paclitaxel, along the portion of the device that will be implanted into the body. Alternatively, the therapeutic agent can be distributed within the material of the device in the section that will be implanted into the body. In these embodiments, no second therapeutic agent is utilized.

Antimicrobials may be used as the therapeutic agent. As used herein, the term ‘antimicrobial’ means any agent that has killing or growth inhibiting effects on one or more microorganisms. Suitable classes of antimicrobials include antibiotics, disinfectants, and antiseptics.

In a preferred embodiment, a therapeutic agent comprises one or more antibiotics having activity against the common microorganisms associated with colonization and/or infection with indwelling cannulae. Examples of suitable classes of antibiotics include tetracyclines, rifamycins, macrolides, penicillins, cephalosporins, other beta-lactam antibiotics, aminoglycosides, chloramphenicol, sulfonamides, glycopeptides, quinolones, fusidic acid, trimethoprim, metronidazole, clindamycin, mupirocin, polyenes, azoles and beta-lactam inhibitors.

Examples of specific antibiotics that may be used in the medical device of the present invention include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin.

The therapeutic agent can comprise a combination of two or more antimicrobials. In these embodiments, the two or more antimicrobials can be located in or on discrete locations on the exterior of the medical device, or the two or more antimicrobials can be blended together and uniformly distributed within or on the surface of the medical device.

Examples of suitable therapeutic agents for use as a therapeutic agent include anticoagulants, antithrombotics, thrombolytics and/or fibrinolytics, and antiproliferatives. The type of agent selected will depend on several factors, including the stage of development of the fibrin sheath at which interference with further development is desired. For example, antithrombotics, such as heparin, hirudin, hirulog and PPACK, directly or indirectly bind thrombin to prevent polymerization of fibrin from fibrinogen, a necessary step in the coagulation process. Anticoagulants, such as the glycoprotein IIb/IIIa inhibitors, attach to platelet receptors and block activation sites, thereby preventing their degranulation and release of serotonin. Other anticoagulants block ADP induced platelet aggregation, such as Ticlopidine and Clopidigrel. Still other anticoagulants such as warfarin and coumadin inhibit the action of vitamin K and the production of coagulation factors. Some anticoagulants, such as aspirin, inhibit platelet aggregation by inhibiting Thromboxane A2.

Thrombolytics and/or fibrinolytics lyse or break down an organized thrombus by activating plasmin, which breaks down fibrin. Examples of suitable thrombolytics and/or fibronolytics include Tissue Plasminogen Activator (tPA), Urokinase, and Streptokinase. Certain matrix metalloproteinases, such as collagenase, can break down the connective tissue of a formed fibrin sheath.

Examples of suitable antithrombotics include heparin, hirudin, hirulog, and PPACK. Examples of suitable anticoagulants include glycoprotein IIbIIIa inhibitors, ticlopidine, clopidigrel, warfarin, coumadin, and aspirin. Examples of suitable thrombolytics and/or fibrinolytics include tPA, recombinant tPA, urokinase, streptokinase, Tenecteplase, Alteplase, Activase, Lysatec, Antistreplase, APSAC, Eminase, Retaplase, Retavase, Hannahpep (Indian King Cobra venom), and Ancrod (Malayan pit viper venom). Examples of suitable matrix metalloproteinases include collagenase. Other suitable agents for the first therapeutic agent include olyeyloxyethyl phosphorylcholine.

A therapeutic agent may comprise an antiproliferative. In a particularly preferred embodiment, the first therapeutic agent 28 comprises natural or synthetic paclitaxel, a derivative of paclitaxel, and/or a paclitaxel pro-drug. Paclitaxel is a natural diterpere product isolated from the Pacific yew tree (Taxus brevifolia). Paclitaxel is a member of the taxane family of terpenes, and was first isolated by Wani et al. (J. Am. Chem. Soc., 93:2325, 1971). Paclitaxel has proven efficacious in the treatment of a variety of neoplasms, and has been approved for use in the clinical treatment of breast and ovarian cancer in the United States.

Paclitaxel functions as an antiproliferative agent; i.e., as an inhibitor of cell replication. It is believed that paclitaxel inhibits replication by inducing an abnormal polymerization of tubulin. This results in stabilization of microtubules and disruption of the cell division process, mitosis. Further, paclitaxel inhibits smooth muscle cell proliferation both in vitro and in vivo.

Paclitaxel can be used in medical devices of the present invention in its basic form, as a derivative (see for example U.S. Pat. No. 6,476,242 to Kingston et al. for 2-Aroyl4-Acyl Paclitaxel Analogs; see also U.S. Pat. No. 6,441,025 to Li et al. for Water Soluble Paclitaxel Derivatives), and/or as a Pro-Drug (i.e., a drug that yields paclitaxel upon action by an appropriate agent, such as a naturally occurring enzyme; see U.S. Pat. No. 6,153,756 to Digenis et al. for Soluble Prodrugs Of Paclitaxel). Also, a preparation of paclitaxel can be utilized. Any suitable preparation can be used, and should facilitate placement of the paclitaxel into or on the medical device of the present invention, and should allow its release from the medical device. Examples of suitable paclitaxel preparations include those described in U.S. Pat. No. 5,681,846 to Triysel for Extruded Stability Formulations For Paclitaxel.

Considerable attention has been directed toward the effects of paclitaxel on a variety of cell types and physiological processes. Paclitaxel may arrest the migration of fibroblasts and smooth muscle cells, thereby reducing or preventing connective tissue formation that often follows fibrin sheath formation. It has also been found to decrease restenosis of human coronary arteries following stent use.

Coatings according to the present invention are radiation curable, or photo-curable as discussed above. Preferred coatings are prepared with a synthetic polymer and a reagent useful as a surface coating agent. Such surface coating agents are discussed in U.S. Pat. No. 6,603,040, which is hereby incorporated by reference in its entirety. This patent discusses the preparation of surface coating agents from a nonpolymeric core molecule comprising an aromatic group, the core molecule having attached thereto, either directly or indirectly, one or more substituents comprising negatively charged groups, and two or more photoreactive species attached to the core molecule through one or more spacer groups, wherein the negatively charged groups are independently selected from salts of organic acids, the organic acids are selected from sulfonic acid, carboxylic acid, and phosphoric acid, the aromatic group is a benzene radical, the photoreactive species are aryl ketones which may be the same or may be different, and the spacer groups each independently comprise a radical of the formula —O—(CH₂)ₙ— wherein n is a whole number equal to at least one. In particularly preferred coatings, the reagent is of the formula 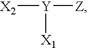
wherein X₁ comprises a first photoreactive species, X₂ comprises a second photoreactive species, Y comprises a nonpolymeric core molecule comprising an aromatic group, and Z comprises at least one charged group.

Examples of suitable charged negative groups include salts of organic acids (e.g., sulfonate, phosphonate, and carboxylate groups), as well as combinations thereof. A preferred charged group for use in preparing coating agents of the present invention is a sulfonic acid salt, e.g., derivatives of SO₃⁻ in which the counter-ion is provided by the salts of Group I alkaline metals (Na, K, Li ions) to provide a suitable positively charged species.

In a preferred embodiment, the core is provided as the residue of a polyhydroxy benzene starting material (e.g., formed as a derivative of hydroquinone, catechol, or resorcinol), in which the hydroxy groups have been reacted to form an ether (or ether carbonyl) linkage to a corresponding plurality of photogroups. In one embodiment, a coating agent of this invention further comprises one or more optional spacers that serve to attach a core molecule to corresponding photoreactive species, the spacer being selected from radicals with the general formula: —O—(CH₂)ₙ—, and —(C₂H₄O)ₘ—C₂H₄O—, wherein n is a number greater or equal to 1 and less than about 5, and m is a number greater or equal to 1 and less than about 4.

In a particularly preferred embodiment, such coating agents are selected from the group 4,5-bis(4-benzoylphenylmethyleneoxy) benzene-1,3-disulfonic acid di(potassium and/or sodium) salt, 2,5-bis(4-benzoylphenylmethyleneoxy) benzene-1,4-disulfonic acid di(potassium and/or sodium) salt, 2,5-bis(4-benzoylphenylmethyleneoxy) benzene-1-sulfonic acid monopotassium and/or monosodium salt. Substitution of carboxylic or phosphoric groups for the sulfonic groups also yields preferred coating agents yielding coatings of great lubricity and hydrophilicity.

These compounds may be combined with synthetic monomers to yield such coatings. Preferred monomers include polyacrylamide, sulfonic acid-substituted polyacrylamide, polyethylene glycol, polyvinyl alcohol, polyvinyl pyrrolidone, silicone monomers, and quaternary-amine substituted polyacrylamide. Other chemical species will also yield hydrophilic coatings, such as alginic acid, hyaluronic acid, pectin, mono- and di-saccharides, heparin, glycogen, chitosan and cellulose. Coatings may be prepared by suitable combinations of the coating agents as described above, the monomer or other chemical species, and a suitable solvent. After preparation of the coating, a suitable dilute solution in water may be prepared and applied to desired medical devices. The method of application may include dipping, spraying, or other convenient desired method. The coating may then be cured by exposing the coating to suitable radiation or photo-energy, such as a UV lamp or other source of suitable photoinitiating activity.

It should be understood that the use therapeutic agents and hydrophilic coatings in catheters is not limited to the applications discussed above. Thus, medical devices with therapeutic agents and hydrophilic coatings may desirably be used in any of a number of other applications, such as for nephrostomy drainage and central venous access. The ease of insertion, the lower chance of infection and complications, and the lower size of catheters and other medical devices will allow a great many applications and embodiments of the invention.

The details of the construction or composition of the various elements of the hydrophilic coated medical device not otherwise disclosed are not believed to be critical to the achievement of the advantages of the present invention, so long as the elements possess the strength or flexibility or softness needed for them to perform as disclosed. The selection of such details of construction is believed to be well within the ability of one of even rudimentary skills in this area, in view of the present disclosure, and are within the spirit of the invention and the scope of the claims. It will be understood that no limitation of the scope of the invention is intended by the above description and drawings, which is defined by the claims below.

1. A medical device for at least partial implantation in a patient, comprising:
an elongated member;
a therapeutic agent disposed on an exterior of the elongated member; and
a hydrophilic coating disposed on the elongated member.

2. The medical device of claim 1, wherein the hydrophilic coating at least partly covers the therapeutic agent.

3. The medical device of claim 1, wherein the hydrophilic coating is intermingled with the agent.

4. The medical device of claim 1, wherein the hydrophilic coating is radiation-curable, UV-curable, photoimmobilizable, or photoreactive.

5. The medical device of claim 1, wherein the hydrophilic coating is a PhotoLink® radiation-curable coating.

6. The medical device of claim 1, wherein the medical device is a catheter.

7. The medical device of claim 1, wherein the therapeutic agent is an anticoagulant, an antithrombotic, a thrombolytic, a fibrinolytic, an antiproliferative, an antibacterial, or an antibiotic agent.

8. The medical device of claim 1, wherein the therapeutic agent comprises minocycline or rifampin.

9. The medical device of claim 1, wherein the therapeutic agent comprises at least two therapeutic agents disposed on the exterior.

10. The medical device of claim 9, wherein the therapeutic agents comprise rifampin and minocycline.

11. The medical device of claim 4, wherein the hydrophilic coating comprises a reagent useful as a surface coating agent, the reagent having a nonpolymeric core molecule comprising an aromatic group, the core molecule having attached thereto, either directly or indirectly, one or more substituents comprising negatively charged groups, and two or more photoreactive species attached to the core molecule through one or more spacer groups, wherein the negatively charged groups are independently selected from salts of organic acids, the organic acids are selected from sulfonic acid, carboxylic acid, and phosphoric acid, the aromatic group is a benzene radical, the photoreactive species are independently aryl ketones, and the spacer groups each independently comprise a radical of the formula —O—(CH₂)ₙ— wherein n is a whole number equal to at least one.

12. A method of making a medical device, comprising:
forming a tubular member defining at least one lumen;
coating at least an exterior surface of the tubular member with a radiation-curable coating; and
curing the coating.

13. The method of claim 12, wherein the radiation-curable coating is a PhotoLink® coating.

14. The method of claim 12, further comprising coating the exterior of the tubular member with at least one therapeutic agent before the step of coating the exterior surface.

15. The method of claim 12, wherein the tubular member is formed with a therapeutic agent is dispersed within the tubular member.

16. A medical device for at least partial implantation in a patient, comprising:
a tube member defining a lumen;
a mixture of rifampin and minocycline distributed throughout at least a portion of the tube; and
a radiation-curable hydrophilic coating on an exterior of the device.

17. A method of making a medical device, the method comprising:
forming a tubular member defining at least one lumen;
coating at least an exterior surface of the tubular member with a radiation-curable coating; and
curing the coating, wherein the coating further comprises a reagent useful as a surface coating agent, the reagent having a nonpolymeric core molecule comprising an aromatic group, the core molecule having attached thereto, either directly or indirectly, one or more substituents comprising negatively charged groups, and two or more photoreactive species attached to the core molecule through one or more spacer groups, wherein the negatively charged groups are independently selected from salts of organic acids, the organic acids are selected from sulfonic acid, carboxylic acid, and phosphoric acid, the aromatic group is a benzene radical, the photoreactive species are aryl ketones that may be the same or may be different, and the spacer groups each independently comprise a radical of the formula —O—(CH₂)ₙ—, wherein n is a whole number equal to at least one.

18. The reagent of claim 17 wherein the reagent is of the formula 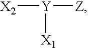
wherein X₁ comprises a first photoreactive species, X₂ comprises a second photoreactive species, Y comprises a nonpolymeric core molecule comprising an aromatic group, and Z comprises at least one charged group.

19. The reagent of claim 18 wherein the Y group comprises a benzene radical.

20. The reagent of claim 19 wherein the charged groups Z are selected from sulfonic acid, carboxylic acid, and phosphoric acid.

21. The reagent of claim 19 wherein the photoreactive species of X₁ and X₂ are aryl ketones, and X₁ and X₂ may be the same or may be different.

22. The reagent of claim 21 wherein the aryl ketones are selected from the group consisting of acetophenone, benzophenone, anthraquinone, anthrone, and anthrone-like heterocycles, and their substituted derivatives.

23. The reagent of claim 17 wherein each aryl ketone is selected from the group acetophenone, benzophenone, anthraquinone, anthrone, and anthrone-like heterocycles, and their substituted derivatives, and the aryl ketones may be the same or may be different.

24. The reagent of claim 17 wherein the spacer groups each independently comprise a radical of the formula —(C₂H₄O)ₘ—C₂H₄O— wherein m is a whole number equal to at least one.

25. The medical device of claim 17, wherein the medical device is selected from the group consisting of a wire guide, a catheter, a stent, a cannula, a venous access catheter, a central venous access catheter, a binary drainage catheter, a suprapubic urinary drainage catheter, a gastrostomy catheter, a dialysis catheter, and an arterial catheter.
